(19) United States
(12) Patent Application Publication
(10) Pub. No.: US 2006/0223091 A1
(43) Pub. Date: Oct. 5, 2006

(54) Microarray and methods of using same

(76) Inventor: Mei Xu, Worcester, MA (US)

Correspondence Address: MIRICK, O'CONNELL, DEMALLIE & LOUGEE, 100 FRONT STREET, WORCESTER, MA, 01608, US

(21) Appl. No.: 11/368,884

(22) Filed: Mar. 6, 2006

Related U.S. Application Data
(60) Provisional application No. 60/658,442, filed on Mar. 4, 2005.

(51) Int. Cl. C12Q 1/68; C12M 1/34

(52) U.S. Cl. 435/6; 435/287.2

(57) ABSTRACT

A DNA microarray device having probes immobilized to the microarray surface, in which targets to be detected using the microarray are exposed to the probes to hybridize therewith. There are provided on the microarray a quantity of a group of perfect match probes for a target of interest such that the molar ratio of each such probe to the target fragment is at least about 20:1.

### CROSS REFERENCE TO RELATED APPLICATION

This application claims priority of Provisional application Ser. No. 60/658,442, filed on Mar. 4, 2005.

### FIELD OF INVENTION

This invention relates to DNA microarray technology.

### BACKGROUND OF THE INVENTION

Microarrays, which allow massive hybridizations to thousands of genes in parallel, can be a powerful tool, shedding light on cellular processes by identifying groups of genes that appear to be co-expressed. See Joseph L. DeRisi, Vishwanath R. Iyer, and Patrick O. Brown. Science Oct. 24, 1997; 278: 680-686. There are two categories of microarrays: cDNA arrays and oligo nucleotide arrays. cDNA arrays are cheaper, and the probe sets can be easily customized. Usually they are in a “one gene, one probe” format, and can often be prepared in house. The main drawback is nonspecific cross hybridization occurrence, and difficulty differentiating. For genome-wide gene transcriptional profiling, cDNA arrays have limited use. A few early projects, including analyses of the yeast cell cycle (Spellman, P. T. et al. Mol. Biol. Cell 9, 3273-3297 (1998) and Cho, R. J. et al. Mol. Cell 2, 65-73 (1998). Golub, T. R. et al. Science 286, 531-537 (1999)) and classification between two forms of leukemia (Golub, T. R. et al. Science 286, 531-537 (1999)) were successfully confirmed with classification, because these projects used a hybridization pattern in which accurate measurements and comparisons of the individual gene expression levels are not needed.

Oligo-nucleotide arrays are often available commercially. A typical example is the Affymetrix “GeneChip”™. The GeneChip™ uses multiple probe pairs, called a probe set, to detect each single gene. A perfect match (PM) and a mismatch (MM) probe define a probe pair. After hybridization, an average difference

$$( \frac{1}{n} {\sum }_{i = 1}^{n} ( P {M}_{i} - M {M}_{i} ) )$$

is calculated as an indicator of the relative gene expression level. Using shorter probes may improve the sensitivity, and multiple probes should enhance the confidence of the measurements.

Unfortunately, despite that some metagenes have been found using microarrays, the increased abundance of data has not substantially improved the poor reproducibility of data and the accuracy of the results. The data sets generated from identical samples can vary drastically. The following discussion demonstrates that in large scale or high throughput hybridization data generation and the data processing and analysis, chemical equilibrium and thermodynamics play a key role. If basic chemical thermodynamics laws are violated in any step of the process, variations are introduced that make it impossible to acquire valid and comparable data, a problem which cannot be corrected by data analysis. Affymetrix GeneChip technology is an appropriate example of a microarray because it is a good device and is a more complicated working platform than other arrays, allowing the discussion herein to cover many relevant questions relating to microarray technology.

It is expected that DNA microarrays will allow researchers to acquire knowledge such as the number of genes that are expressed in a cell and at what level each gene is expressed. Because they generate high throughput data, microarrays are said to be capable of determining a global gene expression spectrum, screening differential gene expression, and even decoding the gene expression regulation network for a cell or a specific cell population. However, despite the fact that thousands of papers have been published on the subject, microarray data interpretation remains a challenge; the same sample can give widely different results when different microarrays are used, and the same data may result in different results when different data interpretation software is used.

Researchers are attempting to depict the gene expression regulation network by using data mining technology, such as hierarchical clustering analysis. It is expected that the knowledge acquired by these types of studies can be used to help find disease-causing genes, to drug discovery, or to monitoring medical treatment of diseases at gene expression level, for example. Finding differential gene expression by gene expression profiling using microarray data is not as simple and easy as cell type classification. The reason is that the scanned fluorescence signal intensity data, which has not been converted into gene expression levels, are directly taken as gene expression levels and used as the input to a data analysis programs. In the comparison and gene clustering analysis, the input is actually the signal intensity data, which may represent the hybridization product. The signal intensity data representing the hybridization product has quite a complicated relationship with gene expression level. How the signal intensity data/product represents the gene expression level is very much a chemical thermodynamics question, and is not as simple as a certain fraction.

In the current microarray technology, a comparison of the signal intensity of the hybridization data between/among samples is often performed when screening the differential gene expression. Fundamentally, the signal intensity is not necessarily equivalent to the gene expression level and so cannot be simply converted into the gene expression level. Also, the signal intensity is subject to many factors in the hybridization reaction process. This is why the data analysis results are often noisy and inconsistent. Although it is possible to find some meta genes essentially by chance through such comparison analysis, such data analysis methods are obviously insufficient for acquiring a systematic gene expression profile.

In the post genome era, functional genomics has become the center of genomics research. After the sequencing of the 3 billion bases of the human genome is completed, understanding gene expression, regulation and the relationship with the functions of each cell, so as to be capable of altering or manually regulating gene expression to find new disease therapies, is the ultimate goal of functional genomics research. The primary goals of gene expression profiling are to acquire knowledge of how many genes are expressed; at what level each gene is expressed; which genes are co-regulated upon an environment stimuli; or a change of the internal needs, for example. Gene expression profiling largely relies on microarray technology. However, the application of the current DNA microarray technology often cannot supply correct and accurate of cell gene expression information. The “noisy” results have confused many researchers. The hybridization data—the signal intensity that is read from the DNA microarray—is subject to many experimental factors. The data can be non-linearly correlated to the gene (mRNA) level, the comparison results sometimes can be correct but still not accurate—meaning by chance some up- and down-regulated genes could be found but it is unlikely that the fold change will be accurate. Although under certain conditions, the relative signal intensity might indicate higher expression level of a gene, this is not reliable and is often interfered with by cross hybridization. The hybridization data does not necessarily reflect the expression level of the gene. When discussing gene expression profiles on a global scale, such uncertainty of analysis is problematic.

### SUMMARY OF THE INVENTION

This invention in part involves an analysis of microarray hybridization systems with chemical thermodynamics, theoretically clarifying some misunderstandings and looking for answers to some critical questions around this technology, such as the mechanisms and conditions of quantitative measurement of hybridization reactions, the reasons for inconsistency of data and data analysis results and solutions, and manners to analyze the data, for example. A theoretical model for the next generation of microarray is proposed: one that is universal, laying the foundation for microarray technology from array design through the data analysis.

This invention features an improved DNA microarray device comprising probes immobilized to the microarray surface, in which targets to be detected using the microarray are exposed to the probes to hybridize therewith, the improvement comprising providing on the microarray a quantity of a group of perfect match probes for a target of interest such that the molar ratio of each probe to the target fragment is at least about 20:1, which may hold for every probe-target pair of interest. The ratio may be at least about 50:1, or even at least about 1000:1. The ratio may be achieved at least in part by decreasing the target concentration in the sample being tested. The microarray may be incubated at a temperature of about a few degrees below the melting temperature of the hybridized probe-target pair. Multiple probes may be used to measure each single gene. The sample amount may be sufficient such that the target gene with the lowest abundance produces a detectable signal after hybridization.

Also featured is a method of determining the presence of a target sequence in a sample that is exposed to a microarray having perfect match probes coupled to its surface, comprising hybridizing under the same conditions a control sample and a test sample, with a series of perfect match probes available for the target sequence, and then comparing the measurements from both samples.

Further featured is a method of determining the concentration of a target sequence in a sample that is exposed to a microarray having perfect match probes immobilized to its surface, comprising testing the sample and at least two different dilutions thereof under the same hybridization conditions, and comparing the data to determine the target concentration in the sample. This method may further comprise providing multiple probes for each target gene, determining the target concentrations from each probe, and comparing the concentrations to determine a concentration value, and determining the identity of the target by coupling the concentration of the target with multiple physical chemical parameters of the hybridization reaction between each probe-target pair. There may be at least about ten probes for each target gene. The ratio of sample dilutions may be in one example about 1:2:3. There may be at least about ten probes per target gene.

### BRIEF DESCRIPTION OF THE DRAWINGS

Other objects, features and advantages will occur to those skilled in the art from the following description of the preferred embodiments and the accompanying drawings, ill which:

FIG. 1 is a set of graphs detailing the PM data distribution over the entire chip for chips MY30A, MY30B, MY31A and MY31B. In order to make the display easier to interpret, the top 1% of the large values of each chip are omitted.

FIG. 2 is a set of graphs of the pattern of one probe set data from eight chips of four identical samples. X: the probe in order; y: digitized signal intensity from scanner. Four pairs of chips are of four RNA samples, i.e., each pair is duplicates of an identical sample: MY30A/MY30B, MY31A/MY31B, AF0/MY18 and AF6/MY19. MY30A/MY30B and MY31A/MY31B were hybridized in the same cycle; AF0 and AF6 in one cycle; MY18 and MY19 in another cycle of hybridizations.

FIG. 3 is two graphs showing that equal predicted melting temperature of probes does not insure equal binding ability to the hybridization target. Horizontal axis: temperature; vertical axis: degree of dissociation. (a) The computer program predicted Tₘ values may be close to one another within a small range, but are not ensured to be the same; (b) The probes having truly equal Tₘ are not ensured to have the same binding ability at the hybridization temperature. The probes' behaviors are unpredictable.

FIG. 4 is a comparison analysis using different approaches: the Affymetrix Signal and AveR method. For each gene, the ratio RMY31B/MY31A_AffySignal and RMY31B/MY31A_AveR were calculated as: for each gene, RMY31B/MY31A_AffySignal=M31B/M31A (if M31B>M31A)=M31A/M31B (if M31B<M31A); similarly, RMY31B/MY31A_AveR=average(Σ(RM31B/M31A)) (if M31B>M31A)=average(Σ(RM31A/M31B)) (ifM31B<M31A). x: ratio range, y, percentage of probe sets. RMY31B/MY31A_AveR<1.2 covers 97.88%, RMY31B/MY31A_AveR<1.3 covers 99.67% probe sets of the entire chip. The maximum value is 1.78. In the contrast, RMY31B/MY31A_AffySignal<1.2 covers 38.36%, RMY31B/MY31A_AffySignal<1.5 covers only 61.08% of the probe sets. The remaining nearly 40% probe sets are spread over a wide range from 1.5 to 184.

FIG. 5 is a diagram of the Southern hybridization method. The target in the DNA samples is separated from the mixture by electrophoresis (1). An abundance (over amount) of probes can detect almost all targets in a sample (2). Non-specific sequence that can hybridize to probe A exists in both sample 1 and sample 2, but catch fewer probes. A: over amount of probes are applied to the hybridization system. B: the target in the sample 1 and sample 2. C: Non-specific sequence. After optimization of the hybridization temperature, the nonspecific product [AC]_{Eq} is minimized while the specific product [AB]_{Eq} can be a clearly visualized band left on the film.

FIG. 6 is a diagram of the relationship between the hybridization signal intensity and the gene expression. A1 through A4: probes immobilized on the solid phase. [A1]₀=[A2]₀=[A3]₀=[A4]₀. The different shapes represent different targets sample solution at different abundances, and are in the order of [B1]₀>[B4]₀>[B3]₀>[B2]₀. The number of targets hybridized to the represents the product/signal intensity, i.e., [A1B1]_{Eq}=[A4B4]_{Eq} and[A2B2]_{Eq}=[A3B3]_{Eq}. In addition, it is assumed that [B1]₀>[A1]₀, [B4]₀>[4]₀, [A2]₀>[B2]₀ and [A3]₀>[B3]₀.

FIG. 7 illustrates the conversion X_{B} compared to the baseline of [A]₀/[AB]_{Eq}=1000 under different equilibrium constant K. x axis: the ratio of [A]₀/[AB]_{Eq}; y axis: the percentage of change in X_{B} when comparing to X_{B([A]}_{₀}_{/[AB]}_{_{Eq}}₌₁₀₀₀₎. The value of “y” is calculated as:
(X_{B}−X_{B([A]}_{₀}_{/[AB]}_{_{Eq}}₌₁₀₀₀₎)/X_{B([A]}_{₀}_{/[AB]}_{_{Eq}}₌₁₀₀₀₎.

FIG. 8 is an example of an effect of the choice of hybridization temperature. Hybridization is conducted at temperature T. More cross hybridization would occur upon lowering T to T1, and less hybridization would occur for most probes upon raising T to T2. The different vertical dotted lines indicate different melting temperatures for different probes: from left to right Tₘ1 for probe 1, Tₘ2 for probe 2, Tₘ3 for probe 3, Tₘ4 for probe 4, Tₘ5 for probe 5, Tₘ6 for probe 6. The dissociation graphs of probes are also shown and are in the same order as the melting temperature lines, from left to right.

FIG. 9 comprises four diagrams of cross hybridization in DNA microarray hybridization. A1, A2, A3 and A4: different probes. B1, B2, B3, and B4: different target sequences. A target sequence may hybridize to different probes beside the specific one (1). A probe may exclusively hybridize to only one target (2), or mainly to the specific target, with minor cross hybridization (3). The signal intensity could be mainly produced by cross hybridization (4)

FIG. 10 gives two examples in which the signal intensity data of Perfect Match (PM) and the Mismatch (MM) probes is shown. The lines represent the probes in the order in PM and MM. Both PM and MM data display a steady upward trend as sample concentration increases.

FIG. 11 shows examples in which cross hybridization occurs easily at lower mole ratio of probe-target[A]₀/[B]₀(1), and can be reduced by change the lifting the ratio. The signal intensity produced by specific hybridization is shown as the darkest squares, while the signal intensity produced by non-specific cross hybridization is shown by lighter squares. In example (1) there is too much target, while the amount of target is reduced (2) in example (2) so as to satisfy the condition of [A]₀>>[B]₀

FIG. 12 is a simulation of the impact of the ratio [A]₀/[B]₀ on the conversion rate of B under different given K values between 2 to 100. The equilibrium constant K of hybridization reaction is supposed to be larger than 2.

FIG. 13 is a bar chart showing an example of a possible composition of the signal from one probe. Light color represents the signal intensity generated by the specific target. The dark and white represent the signal intensity produced by cross hybridization.

FIG. 14 is a matrix of microarray data for computing the absolute expression level of one gene.

### DESCRIPTION OF THE PREFERRED EMBODIMENTS

Part I: Theoretically Modeling Microarrays Using Chemical Equilibrium and Thermodynamics

1. Chemical Equilibrium and Chemical Thermodynamics Issues Have a Profound Impact on Microarray Data.

Microarray technology is based on the nature of nucleotides that two complementary strands hybridize with each other by hydrogen bonds. Nucleotide hybridization is a reversible process. To simplify the following discussions, the letter “A” is used to represent the mole density of a probe, “B” the target sequence and “AB” the product of hybridization reaction. A hybridization reaction is then expressed as: 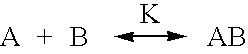
At equilibrium the hybridization product [AB]_{Eq} can be expressed as:
[AB]_{Eq}=K[A]_{Eq}[B]_{Eq} (1)
If [A]₀ is used to represent the initial probe density; [A]_{Eq} and [B]_{Eq} the mole concentrations of the free probes and the free target sequence at equilibrium respectively, then [AB]_{Eq}, could also be written as

$${[ A B ]}_{Eq} = K ( {[ A ]}_{0} - {[ A B ]}_{Eq} ) * {[ B ]}_{Eq} i . e . , \\ \begin{matrix}K = \frac{{[ A B ]}_{Eq}}{( {[ A ]}_{0} - {[ A B ]}_{Eq} ) * {[ B ]}_{Eq}} & ( 2 )\end{matrix}$$

Using formula (2), we can obtain a series of K_{—}_{ⁱ} for the series of reactions between a probe set A₁ through Aᵢ and the target sequence B. The “_{—}_{ⁱ}” labels the probes in order.

$${K}_{- i} = \frac{{[ {A}_{i} B ]}_{Eq}}{( {[ {A}_{i} ]}_{0} - {[ {A}_{i} B ]}_{Eq} ) * {[ B ]}_{Eq}}$$

When [Aᵢ]₀ is known and fixed for all the probe set members, and [Aᵢ]₀>>[B]₀ hence certainly [Aᵢ]₀>>[AᵢB]_{Eq}, [Aᵢ]₀−[AᵢB]_{Eq}≈[A]₀. The following expression is then valid for the whole probe set:

$${K}_{- i} = \frac{{[ {A}_{i} B ]}_{Eq}}{{{[ A ]}_{0} [ B ]}_{Eq}} \qquad ( 3 )$$

Now the denominator is approximately equal to every probe of the probe set. If K is available, it would be very easy to calculate [B]_{Eq} and then [B]₀. Unfortunately K is unknown for any probe. But formula (3) still supplies the most important information: the underlying internal relationships among a probe set chip data:
K_{—}_{¹}:K_{—}_{²}:K_{—}_{³}: . . . :K_{—}_{ⁱ}=[A₁B]_{Eq}:[A₂B]_{Eq}:[A₃B]_{Eq}: . . . :[AᵢB]_{Eq} (4)
The probe set data form a fixed pattern, which is constructed of a series of chemical equilibrium constants, K_{—}_{ⁱ}. In chemical thermodynamics, when a reaction system (all the reactants including the solvents) is determined, K is a function of temperature. In other words, probe set data of the same target sequence in different samples produced at the same temperature will display the same pattern, providing that the target sequence exists. The pattern expressed in formula (4) contains the most important information about the target gene that microarray data contains. It not only allows one to judge if the target sequence exists or not, but also supplies the basis for a comparison of the abundances of the taroet sequence in different samples.

The relationship between K and T, expressed in a simplified fashion, is:

$${K}_{- i} = {ⅇ}^{- \frac{Δ {G}_{- i}^{*}}{RT}} \qquad ( 5 )$$

Here ΔG°, the Gibbs free energy of the reaction system (between A and B), is also a quadratic polynomial function of temperature T, R is a constant. Due to these facts, the dependency of K to T is more complicated than an exponential function. Here we only need to know that the dependency of K to T is nonlinear for now. The impact of a shift of temperature ΔT on each K_{—}_{ⁱ} is different depending on the dependency of each ΔG°_{—}_{ⁱ} to temperature, while ΔG°_{—}_{ⁱ} is a thermodynamic feature parameter of the chemical compound's nature. Each reaction system between a probe molecule and its target sequence molecule has its own ΔG°_{—}_{ⁱ}.

In many microarray laboratories/facilities, the temperatures of hybridization and post-hybridization washing of the Affymetrix GeneChip system have been set at their operating temperatures, which is usually kept constant. However, this does not insure that the data are always generated at exactly the same temperature. A heating incubator works using a heating cycling: heating→reach the upper temperature limit→stop heating→temperature declines→restart heating→ . . . . The hybridization oven, based on the investigation of some Affymetrix GeneChip core facilities, can have visible fluctuations of ±0.1° C. on the thermometer screen. When hybridization is stopped at the upper limit of temperature in the heating cycle, the data value will be lower, and vice versa, to different degree. There are about 200,000 PM probes on a recent version chip (GeneChip MG_U74, HG-U95). On HG_U133A_plus, there are 600,000 PM probes. If MM data is included, the number is about doubled. There are as many Ks as PM probes. A minor temperature shift causes every one of the 200,000 probes reactions to shift differently in terms of their thermodynamics features. The equilibrium of the entire chip shifts. The data on the whole chip becomes truly “unruly”. If an estimated 3,000 to 5,000 genes are expressed in a cell, at most 48,000 to 80,000 probes are responsive to the expressed genes (each gene has 16 PM probes). When temperature shifts ΔT° C. from the set temperature, the hybridizations of this fraction of the probes, except some of those that have infinitely small K, follow the rule of formula (5). The remaining 120,000 to 152,000 PM probes are divided into two classes depending on whether or not non-specific cross hybridization occurs to the probe: if yes, formula (4) also works for the reactions between the probes and the non-specific target sequences. Otherwise no matter how the theoretical K_{—}_{ⁱ} changes, data would remain zero or at the background level due to the fact that [B]_{Eq} is null as indicated in formula (3). No reaction occurs to these probes. The signals read from these cells are only the background produced by the adsorption by the chip surface. The changes of the signal intensities of these probe data follow the thermodynamics of the adsorption process of the glass surface to the fluorescence materials and nucleotides in the solution.

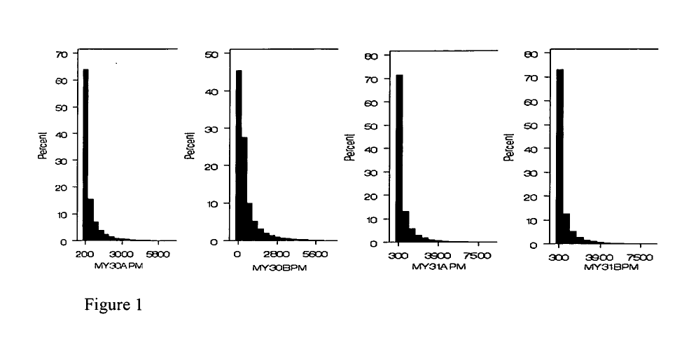

Detailed knowledge about the transcripts abundance per cell is not yet available. Information obtained from the Series Analysis of Gene Expression (SΔGE) indicates that across all the eukaryotic cell types, fewer than 100 transcripts account for 20% of the total mRNA population, each being present between 100 to 1,000 copies. A further 30% of the transcriptome comprises several hundreds of intermediate frequency transcripts, with between 10 to 100 transcripts per cell. The remaining half of the transcriptome is made up of tens of thousands of low abundance transcripts. Thus most transcripts contribute less than 0.01% of total mRNA. Affymetrix GeneChip data also shows a similar distribution wherein most probe data are very closely focused at lower value range as shown in FIG. 1.

From this perspective, it is not hard to understand that things become worse when trying to normalize the data that are generated in different hybridization cycles for clustering analysis. In many microarray laboratories/core facilities, Affymetrix SUITE output “.chp” data using “target intensity”, may be set at 1,500 or some other number. Every “average difference” of each chip is multiplied by a scaling factor so as to make the average of the intensities (Affymetrix SUITE uses the average differences) of the chip to be 1,500. Alternatively, many microarray data analysis programs use a “mean or median centering” method for the normalization of a group of chips, i.e., multiplying every value on the array with a “scaling factor” so as to achieve “equal brightness”. The scaling factor is calculated based on the data of a “baseline” chip.

This type of data processing violates the rule of formula (4). In order to normalize the data sets that are generated in different hybridization cycles without strict thermodynamics condition control, the thermodynamics constants of each component are required to get the dependencies of every K to T for each pair of probe-target reactions. That is, if not absolutely impossible, far from currently realistic. The data sets that are generated in the same hybridization cycle cannot be “normalized” by a “mean or median centering method” neither for correcting the variations caused by the differences of the RNA sample amount being applied to chips. As discussed above, the probes that hybridized to no targets should not be multiplied by a “scaling factor”. The portion of these probes on the chip is probably larger than those that hybridize, especially when the chip contains more genes as in the genome wide chips such as HG_U133A_plus.

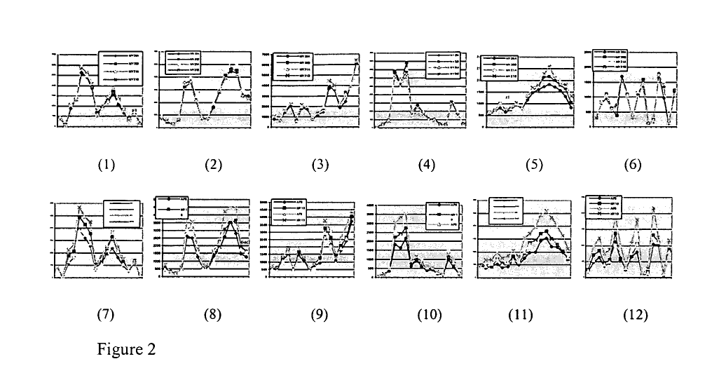

The following examples demonstrate that duplicate data of identical samples that were produced in the same hybridization cycle are more identical than those that were generated in different cycles. FIG. 2 displays the raw data (contained in the .cel file) of a few probe sets from MG-U74A chips. The samples are RNA from uteri of mice (MY. Yao, etc, Mol Endocrinol. 2003 April; 17(4): 610-27. Epub Jan. 9, 2003). In the upper six panels, the four chips are of two samples generated from the same hybridization cycle. The duplicate data show more consistency than the lower six panels of another two samples, which were generated in different cycles. It is obviously better to do hybridization in the same cycle than in different cycles.

2. Misunderstanding Regarding Computer Predicted Melting Temperature of Probes

One of the points in GeneChip design is that all the probes on the chip are designed to have the same or similar melting temperature (Tₘ) and this makes probes on the entire chip have “similar” binding affinity to their target sequences. This is a misunderstanding about melting temperature. Strictly speaking, melting temperature is a state parameter derived from a chemical thermodynamics concept: the degree of dissociation (α). In a reaction in which one molecule reactant creates more than one product, e.g., 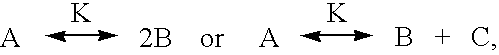
the degree of dissociation of A is defined as: at equilibrium, the amount of dissociated A divided by the initial amount of A. In the case of 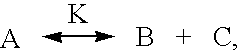
If [A]₀ is the initial concentration of A, [B]_{Eq} and [C]_{Eq} the concentration of B and C at equilibrium, α=([A]₀−[B]_{Eq})/[A]₀=([A]₀−[C]_{Eq})/[A]₀. Applying this concept to a double strand DNA, the temperature at which α=0.5 is defined as Tₘ of that DNA. The DNA hybridization is the reversal of the reaction 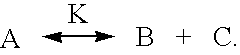
One can see that (t is linked to the chemical equilibrium constant K. If the initial concentration of A is [A]₀, α=0.5 indicates the temperature at which

$$K = \frac{( 0.5 ) [ {A}_{0} ] * {( 0.5 ) [ A ]}_{0}}{{( 0.5 ) [ A ]}_{0}} = {0.5 [ A ]}_{0} ,$$

for the double strand DNA dissociation reaction. How the temperature T and the Gibb's free energy ΔG° determines K is described in detail elsewhere herein. Here T is determined. The ΔG° of each pair of probe-target is definitely different because each pair of the molecules are different from the others.

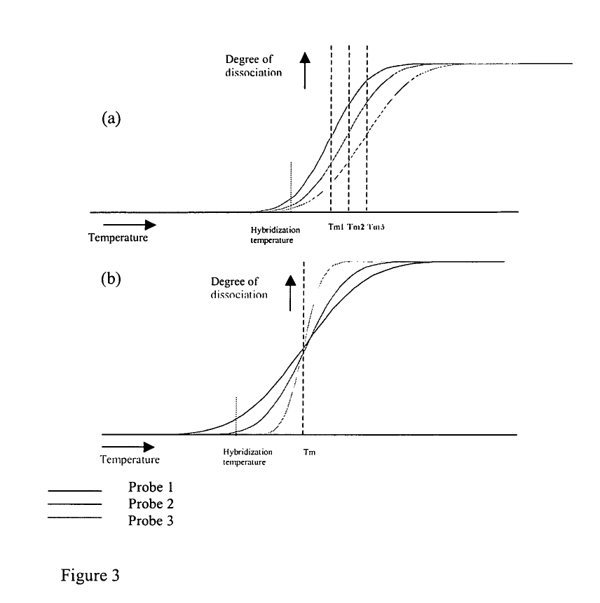

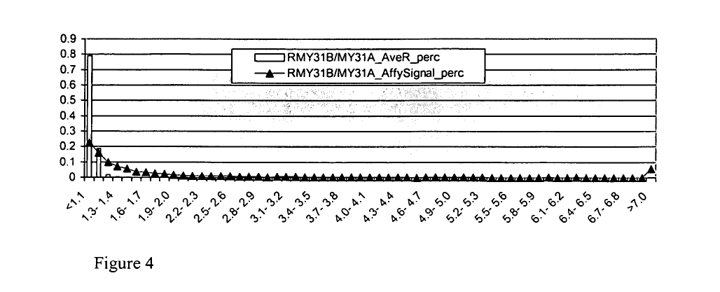

Around the definition of Tₘ, there are a few things to address: 1) the temperature is for that specific double strand DNA molecule with specific sequence and length, for example 25 bp. In microarray hybridization, the target sequences are always different fragments of mRNA, in which the lengths are unknown after the fragmentation reaction, not necessarily even close to 25 bases long. The melting temperature between a 25 base long probe and an unknown mRNA fragment is not necessarily equal to the predicted Tₘ. 2) Melting temperature is a state parameter, indicating that at this specific temperature, the double strand DNA will exist in a specific state, i.e., the α=0.5. It is clear that α is actually the indicator of binding affinity of the two complementary strands. Outside of this temperature we can never predict what the α will be, because the thermodynamics parameters, ΔG°, which are necessary for the prediction, are unavailable. 3) The Tₘ predicted by computer program is based on simulation functions, which can be used for a rough assessment but is not accurate. That each Tₘ is different from the others is absolute. Some may be equal by chance, but not as determined by thermodynamics, as illustrated in FIG. 3(a); even the probes that do have equal Tₘ do not necessarily have equal binding power at hybridization temperature as shown in FIG. 3(b).

In summary, Tₘ is a thermodynamic parameter of the specific molecule. If the temperature is reduced to a low level, all the probes would have hybridized to the target sequences, if the targets do exist. It is still impossible for a probe set to acquire equal data, not only because the fragments are of different length and different labeling integration, but also because the potential binding affinities of different probes are different. The probes having higher potential binding affinity will hybridize to other sequences that are only partial complements in sequence. Regarding the binding ability of the probes of a probe set on a chip to their hybridization target sequences, the binding affinities are absolutely different: no homogeneity is insured. The same or similar binding affinity happens only by chance.

It is well known that the percentage of G+C in a DNA sequence is often used to assess how tightly the two single strands of a DNA molecule bind with each other. The more G and C in the sequence, the tighter the binding and the higher temperature that is required to de-nature the double strand DNA, meaning a higher Tₘ. Tₘ has been used as a parameter to predict the hybridization temperature in the probe design, but not for DNA probe binding affinity at the hybridization temperature. Usually hybridization can be performed at a temperature that is 5-10° C. below the Tₘ (J. Sambrook, et al., <<Molecular cloning>>Cold Spring Harbor Laboratory Press, 1989). When accurately quantitatively measuring thousands of genes simultaneously was not the goal in the experiment, this mechanism works well enough and satisfactorily carries out the task. Also when only a single target sequence (one gene) in multiple samples on an electrophoresis gel were tested (as traditional Northern hybridization) and compared using only one probe and the goal is semi-quantitative measurements, the data and the comparisons are valid.

3. Data Mining of Microarray Data Should Integrate Chemical Thermodynamics Into the Program.

Such incorrect understanding about Tₘ has in part caused misguided efforts in the past years in attempting different statistical approaches for GeneChip data analysis. Among these are: Affymetrix SUITE (versions 4 and 5), dCHIP (C. Li & H Wong. Genome Biol. 2. 0032,1-0032,11. (2001)) and RMA (T. Speed, http://stat-www.berkeley.edu/users/terry/zarray/Talks (June 2002)). These approaches have used a “trial and error” strategy with different statistical methods. None really reasoned why the approach was chosen or why one is better than the others. These approaches seek an “average difference”(Affymetrix SUITE) or “log average”(RMA) to produce a single value from a probe set of data, and use the single value to represent the probe set data, or modify data values (SUITE version5 modifies MM to erase negative signal values), or generate a statistical most probable value (dCHEP, Model-base array data analysis). An average or mean value of a group of data—statistically the “most probable value”, and the standard deviation are parameters used to describe a group of random data and its value distribution. GeneChip data is absolutely not random, however, as the chemical equilibrium constants strictly stipulate each probe's behavior. The chip probe set data best illustrate this point. Taking FIG. 2 (image 6) as an example; the probe set in four chips all display the same or similar pattern. The differences in binding affinity among probes are not random variations but ruled by thermodynamics. The data statistics are summarized in table 1.

TABLE 1
Summary of the numerical data of a PM probe set

| statistics | MY30A | MY30B | MY31A | MY31B | AF0 | MY18 | AF6 | MY19 |
|---|---|---|---|---|---|---|---|---|
| max | 1795.5 | 1905.0 | 1864.0 | 1953.0 | 1111.0 | 1380.5 | 1238.8 | 2170.8 |
| min | 105.0 | 111.3 | 93.0 | 92.0 | 89.0 | 79.0 | 97.0 | 126.3 |
| max/min | 17.1 | 17.1 | 20.0 | 21.2 | 12.5 | 17.5 | 12.8 | 17.2 |
| max − min | 1690.5 | 1793.7 | 1771.0 | 1861.0 | 1022.0 | 1301.5 | 1141.8 | 2044.5 |
| average | 836.6 | 878.5 | 913.0 | 853.4 | 543.6 | 649.3 | 616.9 | 978.0 |
| stdev | 541.7 | 599.5 | 583.8 | 575.2 | 344.4 | 424.0 | 379.4 | 620.6 |
| stdev/average | 64.75% | 68.23% | 63.94% | 67.40% | 63.36% | 65.30% | 61.50% | 63.45% |

max and min: the maximum and the minimum value of the PM data set.
average: the average of the entire PM probe set data;
stdev: the standard deviation of the entire PM probe set;
stdev/average: the standard deviation of the PM probe set data divided by the average of the PM probe set data.

Within a probe set, the data can differ from each other more than 21 fold (see the “max/min” row). The standard deviation of PM probe set data in all eight chips are all larger than 60% of the mean value. If one looks at the entire chip, the average of the Stdev/average of probe sets (table 2, the column of average_(Stdev/average)) is 96.33˜96.38%.

TABLE 2
The Standard deviation/average of PM set data over the entire chips.

| Chip | Ave_(Stdev/average) | StdDev_(Stdev/average) | Min_(Stdev/average) | Max_(Stdev/average) |
|---|---|---|---|---|
| 30A | 93.33% | 41.33% | 6.08% | 346.60% |
| 30B | 94.70% | 42.53% | 5.61% | 360.07% |
| 31A | 96.38% | 43.76% | 4.47% | 357.95% |
| 31B | 96.03% | 42.92% | 6.46% | 357.06% |

(Stdev/average): the standard deviation of PM data set divided by the average of the PM data set. Using this value we can evaluate how large the standard deviation of probe set is and hence how well the data are centered around the average of the probe set;
ave_(Stdev/average): the average of Stdev/average of the entire chip;
StdDev_(Stdev/average): the standard deviation of “Stdev/average” of the entire chip;
Min_(Stdev/average) and Max_(Stdev/average): the minimum and the maximum values of the “Stdev/average” of the chip.

At the same time, most of the PM data values within a chip are very closely centered at a very narrow range. As shown in FIG. 1, of the 200,000 PM data for 12,000 genes, 80% are centered in a very low range (<800˜900).

In summary, in most cases a single value that is calculated from a probe set of data by either of the above methods can hardly correctly reflect the gene expression level or help to find the differences or changes of the gene expression level. MM data are generated by the reactions between unknown sequences and MM probes. MM data are not directly related to the specific gene expression level. The difference of PM-MM or signal (the modified “average difference” of the previous version) can hardly be defined as the “relative indicator of gene expression level” and is even uncertain due to the unpredictable behavior of the MM probes. Most importantly, improperly combining all the data of a probe set into a single value reduces the dimension of the measurements, causing the loss of the most important information—the probe set data pattern, as discussed above.

4. Proposal for a Novel Working Microarray Platform

Below is proposed a novel platform for the next generation of microarray and the algorithms for the data analysis. Based on formula (3):
[A₁B]_{Eq}:[A₂B]_{Eq}:[A₃B]_{Eq}: . . . :[AᵢB]_{Eq}=K_{—}_{¹}:K_{—}_{²}:K_{—}_{³} . . . :K_{—}_{ⁱ}
The microarray should be in the format of “multiple probes for each gene”, i.e., using multiple probes or a probe set to detect each single gene. The probes are PM only, no MM.

(1) Judging if a Gene is Expressed in a Sample

An mRNA's existence can be deduced by two pieces of information: 1) the gene has been known to be expressed in some specific cell populations. Based on an existing genetics database and long years of research, the community is sure that some genes are expressed in certain cells/tissues. We will name this cell/tissue as control sample C; 2) the probe set data pattern of the gene is in both the sample C and the test sample, which will be defined as sample S. Comparing the pattern of the probe set data of the sample S to that of sample C, we can deduce if the gene is expressed or not in the unknown sample: A similar pattern implies yes; otherwise, no. As shown in the lower panels (panels 7-12) of FIG. 2, even the probe set data generated in different hybridization cycles still maintain a similar pattern to some extent—not as strict as the ones generated in the same cycle, though. One piece of additional information is the probe set pattern obtained from multiple arrays: a closely similar pattern implies the same sequence exists in different samples. This is complementary but not firm evidence because in the same type of cells or tissue sources, the cross hybridization source sequences and interferences might be similar, too.

(2) Comparison Analysis

Comparison analysis acquires the information of differential gene expression so as to find out the “up-” or “down-” regulated genes and fold changes. In order to acquire accurate comparison results of gene G in control sample C and testing sample S, the two samples must be hybridized in the same cycle.

For test sample S, the probe set data of gene G are:
PM_{—}_{¹}s:PM_{—}_{²}s: . . . :PM_{—}_{ⁱ}s=K₁:K₂: . . . :Kᵢ

For control sample C, the probe set data of gene G are:
PM_{—}_{¹}c:PM_{—}_{²}c: . . . :PM_{—}_{ⁱ}c=K₁:K₂: . . . :Kᵢ

We define R as the ratio of each corresponding probe of a probe set: R=PMₛᵢ/PM_{ci}. Due to the complexity of the chemical reactions, for the probe set for which the target genes exist, there could be several exceptional situations: the K is infinitely small or the target does not exist in both chips, which means that the reaction almost would not occur. If no significant cross hybridization occurred, the data values in both chips would be close to the background. After subtracting the background, the ratio is theoretically zero divided by zero, which is a very uncertain value.

Part II: Design of DNA Microarrays and Experiments therewith on the Basis of Chemical Thermodynamics

1. The Equilibrium of the DNA Hybridization Reaction and Quantitative Measurement of Gene Expression Level.

Distinctive chemical thermodynamics characteristics of DNA microarray hybridization reaction systems are studied herein. The study demonstrates that the data does not directly represent the gene expression level. Replacing the gene expression level with signal intensity in the data analysis programs, when the goal is to determine the gene expression level, thus will not produce correct results. Only after the relationship between the data and the gene expression level is well understood would it be possible to correctly utilize the data. The study herein also supplies chemical thermodynamics basics for proper array design, and proper experimental design.

It is often said that DNA microarray technology is derived from the Southern hybridization method. Hybridization methods utilize the phenomenon that two complementary strands of nucleic acid may bind with each other with hydrogen bonds under certain conditions. For a single hybridization, if “A” represents the probe, “B” the target sequence, and “AB” the hybridization product, then the reaction can be expressed as: 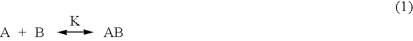
“K” is the chemical equilibrium constant. The [symbol] and “K” in the formula (1) indicates that hybridization is a reversible reaction, i.e., the nucleotide hybridization reaction is conditional and incomplete. At equilibrium, the product of the hybridization can be calculated as:
[AB]_{Eq}=K[A]_{Eq}[B]_{Eq}=K([A]₀−[AB]_{Eq})([B]₀−[AB]_{Eq}) (2)
[ ] represents the molar amount of the components in the reaction system. At the end of the hybridization (usually after multiple hours or overnight in a hybridization oven), it is assumed that the entire complex reaction system has reached equilibrium. In formula (2), [AB]_{Eq} is the amount of the hybridization product at the equilibrium and the [A]_{Eq} and [B]_{Eq} are the moles of free “A” and “B” that are left in the system—A in the solution, B fixed on the hybridization membrane. [AB]_{Eq} is determined by three factors: K, [A]₀ and [B]₀. (To simplify the problem, the concept of “activity” in the chemistry is not explored herein). A variation of any of the three factors will change the product [AB]_{Eq} and hence the signal intensity data value. In Southern hybridization reactions, one can add as many probes as one wants. When [A]₀>>[AB]_{Eb}, it is possible that [AB]_{Eq}≈[B]₀, i.e., the reaction is close to complete. Under such situation, the signal intensity linearly correlates to the product and hence [B]₀. However, it is well known that Southern hybridization is a semi-quantitative method. The requirement for a Southern hybridization experiment is often to answer such questions as: “Is the level of the target B in sample 1 higher than in the sample 2?” An answer like “Yes” or “No” can be perfectly satisfactory.

In some cases, a quantitative result might be desired. Take the following case as an example: there are three samples. The goal is comparing the abundance of B in three samples S1, S2 and S3. Suppose that the real levels of B in the three samples are in the ratio of 2:3:5. When an “over amount” of probe A is poured into the hybridization bag, due to the fact that in the hybridization reaction system [A]₀>>[B]₀ and at proper temperature, the reaction system will be able to let all B become AB, i.e., [AB]_{Eq}≈[B]₀. This means that:
[AB_{S1}]_{Eq}:[AB_{S2}]_{Eq}:[AB_{S3}]_{Eq}≈[B_{S1}]₀:[B_{S2}]₀:[B_{S3}]₀=2:3:5
Since all the samples are loaded onto the same electrophoresis gel, with the electrophoresis being transferred to the same membrane by the same process, the ratio of the amounts of the target in the three bands should not change. Theoretically and practically the quantitative ratio is kept at close to 2:3:5. This result is correct, roughly “accurate” or say, reliable. More often Southern hybridization is used to detect the existence of a specific target. The question thus becomes: “Does B exist in sample 1 or sample 2?” The answer is similarly “Y/N”. The experiment does not even require the result to be [AB]_{Eq} [symbol] [B]₀. In order to reduce non-specific hybridization, the reaction is often not performed under the condition, that a large K_{AB} for the aimed target (such as between A and B in FIG. 1) and smaller K_{AC}, K_{AD}, etc, so as to minimize [AC]_{Eq} and [AD]_{Eq}. The requirement is that the band [AB]_{Eq} is clearly visible with a relatively clean background. 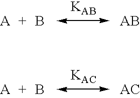
[AB]_{Eq}=K_{AB}([A]₀−[AB]_{Eq})([B]₀−[AB]_{Eq})
[AC]_{Eq}=K_{AC}([A]₀−[AC]_{Eq})([C]₀−[AC]_{Eq})
It is easy to get [AB]_{Eq}>>[AC]_{Eb} when B in the sample has been amplified by PCR after optimization of temperature.

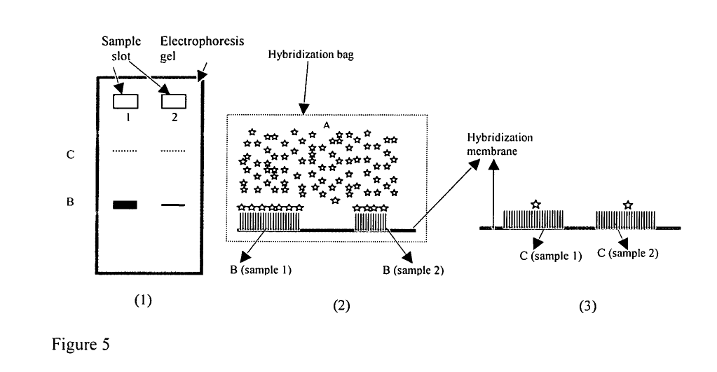

The goal of DNA microarrays is the quantitative measurement of the thousands of genes. In the reversible complex reaction system of a DNA microarray hybridization system, there are thousands of probes fixed on the solid phase while thousands of target sequences are in the mixed sample solution. Probes and targets are mutually exposed to each other. See FIG. 5. For each probe-target pair, by equation (2) there is
[AB]_{Eq}=K_{AB}([A]₀−[AB]_{Eq})([B]₀−[AB]_{Eq})
The goal is to find [B]₀.

Therefore

$${[ B ]}_{0} = {[ A B ]}_{Eq} + \frac{{[ A B ]}_{Eq}}{K ( {[ A ]}_{0} - {[ A B ]}_{Eq} )} \qquad ( 3 )$$

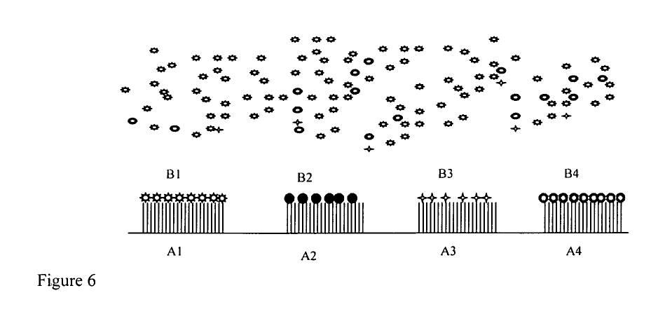

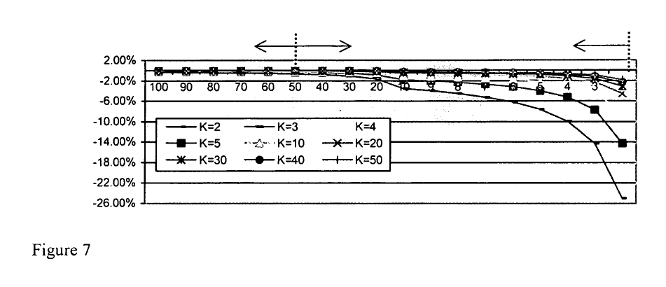

It is easy to see that [B]₀ is not linearly correlated to [AB]_{Eq}. See FIG. 6.

Until now, we have made the observation and clearly demonstrated that DNA microatray technology is not simply an amplified Southern hybridization. The difference between the two categories of technology is not limited to the scale of data, but is more profound. The chemical thermodynamics characteristics of the traditional Southern hybridization method and DNA microarray hybridization are distinctively different. The different goals of the experiments and the distinctive chemical thermodynamics features of the two categories of technologies are summarized in Table 3.

TABLE 3
Comparison of the traditional and microarray hybridization

| Difference | Traditional hybridization | Microarray hybridization |
|---|---|---|
| target location | solid phase | solution |
| target identity | clear | unclear |
| target number | 1, being separated from mixture | hundreds to thousands, in the mixture |
| target-probe relationship | multi(target) to one(probe) | multi(target) to multi(probe) |
| probes location | solution | solid phase |
| over amount probes | not always needed but easy to achieve | always needed but not always satisfied |
| goal | non-quantitative, detection | quantitative measurement |
| numerical data accuracy | not necessary | necessary |

2. The Concept of Conversion Rate in the Reversible Chemical Reaction and Its Importance in the Quantitative Measurement by a Chemical Reaction

In the following discussion by demonstrating that the product of each probe-target pair represents different percentage of the targets in the sample, it is explained why the transformation of the signal intensity into gene expression level is needed in order to acquire the information of gene expression.

From equation (2):

$$K = \frac{{[ A B ]}_{Eq}}{{{[ A ]}_{Eq} [ B ]}_{Eq}} = \frac{{[ A B ]}_{Eq}}{( {[ A ]}_{0} - {[ A B ]}_{Eq} ) ( {[ B ]}_{0} - {[ A B ]}_{Eq} )} -> K = \frac{1}{( {[ A ]}_{0} - {[ A B ]}_{Eq} )} * \frac{{[ A B ]}_{Eq} / {[ B ]}_{0}}{( 1 - \frac{{[ A B ]}_{Eq}}{{[ B ]}_{0}} )} \qquad ( 4 )$$

Let X_{B}=[AB]_{Eq}/[B]₀. X_{B} is called the conversion rate of B at equilibrium, representing the percentage of [B]₀ that had been converted into product AB. For given [B]₀, the more[AB]_{Eq}, the larger the X_{B} is. We also define

$$c = \frac{1}{( {[ A ]}_{0} - {[ A B ]}_{Eq} )} and a = \frac{{X}_{B}}{1 - {X}_{B}} .$$

Then formula (4) is simplified as K=c*a. K is a function of temperature. Assuming that temperature is never changed, then K can be treated as a constant. Hence “c” and “a” are in a relationship of reciprocal. As the ratio of [A]₀/[AB]_{Eq} decreases, “c” increases and “a” decreases. For example, when [A]₀/[AB]_{Eq}=1000/1→[A]₀−[AB]_{Eq}=0.999*[A]₀. If the 0.1/% difference in [A]₀ is tolerable, then we can say that[A]₀−[AB]_{Eq}[A]₀. The “c” would increase 0.1%, or say approximately

$$c \approx \frac{1}{{[ A ]}_{0}} .$$

The conversion rate X_{B} is viewed approximately a constant. If[A]₀/[AB]_{Eq}=100/1, “c” would increase about 1%. The change may cause a minor decrease in “a” and in turn X_{B}. As [A]₀/[AB]_{Eq} decreases to 10/1, for a reaction having K>1, “c” would change more than 11%. The impact of the change of “c” on “a” and hence on X_{B} would become not negligible. As shown in FIG. 3, X_{B} can no longer be viewed as a constant when [A]₀/[AB]_{Eq} reaches a certain number. X_{B} shifts as the ratio of [A]₀/[AB]_{Eq} changes. In the simulation of comparison of the conversion rate to the baseline of [A]₀/[AB]_{Eq}=1000 (when the conversion rate is thought of as nearly a constant, FIG. 3), i.e., the value in the “y” axis is calculated as: (X_{B}−X_{B([A]}_{₀}_{/[AB]}_{_{Eq}}₌₁₀₀₀₎)/X_{B([A]}_{₀}_{/[AB]}_{_{Eq}}₌₁₀₀₀₎. This simulation indicates that in the reversible reaction of DNA hybridization, a change of [A]₀ or [B]₀ or K, jointly or individually, may cause a shift of the equilibrium and change the product [AB]_{Eq}. X_{B}—the conversion rate of B is never a constant. Only when [A]₀>>[AB]_{Eq} can we take [A]₀−[AB]_{Eq}≈[A]₀. Then X_{B} can be viewed approximately as a constant. Under such conditions, the signal intensity of the microarray data is assumed to be nearly linearly correlated to the level of the target in the sample. If a line is drawn at [A]₀/[AB]_{Eq}>=50 (FIG. 7), we see that above 50, the change in X_{B} is minor. While if in a microarray the ratios of probes to targets are below about 50, and particularly below about 10, the variances in X_{B} can cause big problems. See FIG. 7.

On the same array, assume that for any probe Ai, the molar amount [Ai]₀ is equal in the entire array. To different genes, the same data value could represent different meanings because X_{B} could be different. For the same gene in different samples and hybridized to different arrays, if the abundances are different, i.e., [B]₀ is different, certainly [AB]_{Eq} and X_{B} are both different given that [A]₀ is maintained the same for all the arrays. The raw data are not directly comparable. As displayed in FIG. 2, A2-B2 and A3-B3, [A2B2]_{Eq}=[A3B3]_{Eq}, does not necessarily mean that [B2]₀=[B3]₀. In the extreme situation, when [B]₀>=[A]₀ (FIG. 2, A1 and B1, and A4 and B4), the approach that using A to measure B no longer applies. The solution for equation (3) does not have solution for [B]₀. In such a situation, no matter what the data is, one can never deduce how much [B]₀ there is in the system.

3. The Cross Hybridization Problem in DNA Microarrays

In traditional Southern hybridization, probe and target are in a one-to-one relationship. As soon as the probe identifies the target and it can be visualized, all is set; there is no need to consider what happened or may happen to the other targets. At the most, one might want to consider how to obtain a clean background. When assembling a DNA microarray hybridization reaction system, thousands of probes (fixed on the solid phase) are exposed to the thousands of targets in the sample mixture solution (the number of targets is unknown for any sample to the data). Each target exists at different length, abundances, and sequences. The occurrence of cross hybridization is inevitable. This has always been a big problem for DNA microarray applications.

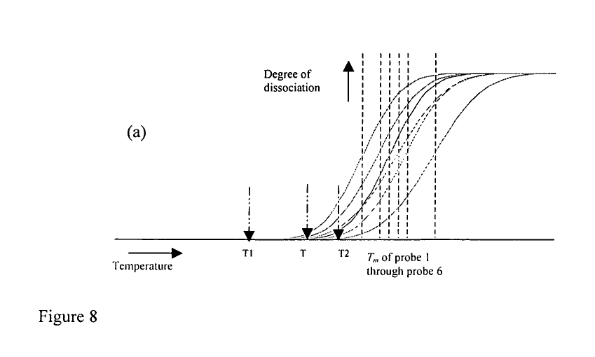

In the traditional Southern hybridization experiment, the optimization is focused on only one probe-target pair. A proper temperature means a relative larger K, allowing specific hybridization to occur and as little as possible non-specific cross hybridization. However, in a DNA microarray there are thousands of probe-target pairs, and each pair has its own melting temperature. It is impossible to optimize the temperature for each pair. One temperature at which most of the target might hybridize would be arbitrarily chosen to be the hybridization temperature (FIG. 8). As a consequence, for some probe-target pairs, the temperature is good enough, such as Tₘ6, while for Tₘ1, it could be sort of “unfair”; if the hybridization temperature was reduced to T1, cross hybridization will be significantly increased.

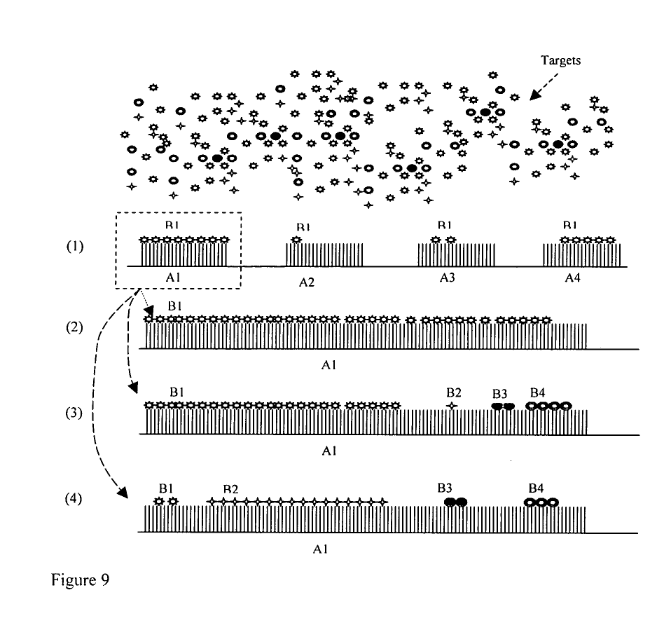

When the abundances of all the targets are equal, i.e., [B1]₀=[B2]₀=[B3]₀=]B4]₀, and[A1]₀>>[B1]₀ and [A2]₀>>[B2]₀ and [A3]₀>>[B3]₀ and [A4]₀>>[B4]₀, we can say that the probability is that the target has the highest chance to hybridize to its probe. Unfortunately, this is not the situation of cellular gene expression. In a cell some genes have as few as one copy per cell, while the abundances of other genes can reach as high as thousands of copies per cell. At the time of reaching equilibrium between the probe and the specific target, the genes with a larger transcription frequency have a larger number of copies left in the solution. The cross hybridization of these genes to the other non-specific probes can result in lower binding affinity (lower equilibrium constant Ks) compared to the specific targets (see FIG. 9), but can still produce non-negligible signal intensities. Especially when the specific targets are in lower abundance or even missing in the sample, the potential non-specific targets with high abundance may become the main source of the signal intensity of that probe (FIG. 9-(4)). It is necessary to point out that the DNA microarray hybridization reaction system is an entire whole, in which the equilibrium is among all the molecules. Each probe-target pair must be considered as part of the entire chemical equilibrium system and cannot be separated from the entirety.

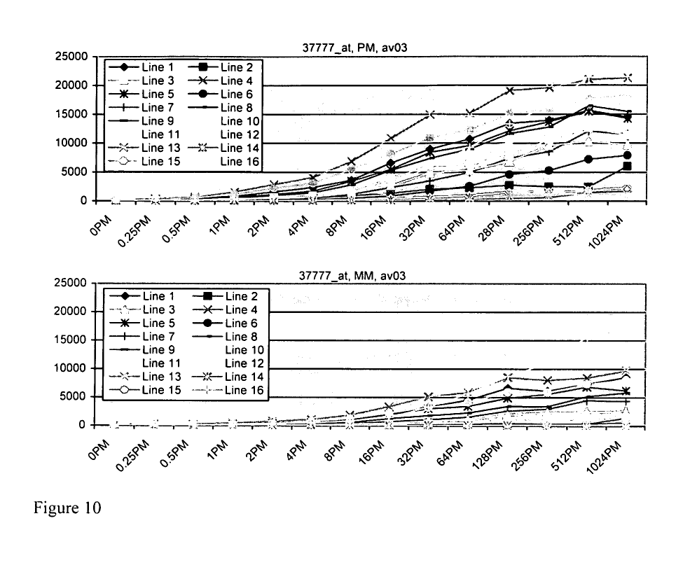

The spike in data of the Latin square experiment shows that cross hybridization is very common. See FIG. 10. The MM data is in the same order of the PM data. With only 14 transcripts and no complex targets, the hybridization is almost surely coming from the specific target sequence. In other words, the “Mismatch” probes produced signal data by cross hybridization to perfect match targets. For clarity we chose only one probe set. In fact, all 14 probe sets that are used in the spike in experiment display similar results. In each MM probe, there is a 4% mismatch in comparison to the corresponding PM probe (one central base out of 25 bases). Considering that 8%, 12%, 16% . . . of mismatches between any probe and any target (not necessarily to the MM probes) on the chip and the drastic variance in the abundances of different genes, it is not hard to understand that cross hybridization would occur to different extents.

4. Designing DNA Microarray and Experiment on the Basis of Chemical Thermodynamics

In the above description, a study of DNA microarray hybridization systems was presented. This establishes that in the current DNA microarray technology, the entire microarray hybridization system is chaotic and full of non-specific hybridizations. However, clues for the improvement of this technology are also available. These are based on fundamental principles of chemical thermodynamics.

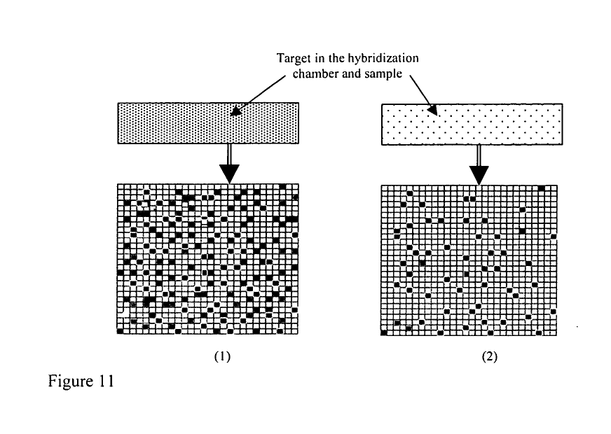

Design rule 1 (see FIG. 11): Establish the condition that [A]₀>>[B]₀, i.e., the molar amount of the probe far more than the target, be satisfied for every probe-target pair. In the reaction of formula (1), when using A to quantitatively measure B, [A]₀>=[B]]₀ is a requirement. When [A]₀>>[B]₀ is satisfied, the conversion rate of X_{B} approximates to constant, i.e., [AB]_{Eq}∝[B]₀. Under such condition, once [A]₀ is available and the hybridization temperature is consistent (i.e., K won't change), the data are comparable. In a DNA microarray, once the hybridization reaction of the gene with the highest abundance also meets the criteria of [A]₀>>[B]₀, much less free target is left in solution at equilibrium. This should significantly reduce the occurrence of cross hybridization. The entire image of the hybridization reaction would be changed from FIG. 11-(1) to FIG. 11-(2).

Design rule 2: The minimum requirement for the sample is the gene that has the lowest abundance can produce a detectable signal by scanning after hybridization. If for example it is Inown that one gene has an abundance of one copy per cell, then if the signal produced by hybridization of this gene is detectable, the sample amount used is sufficient.

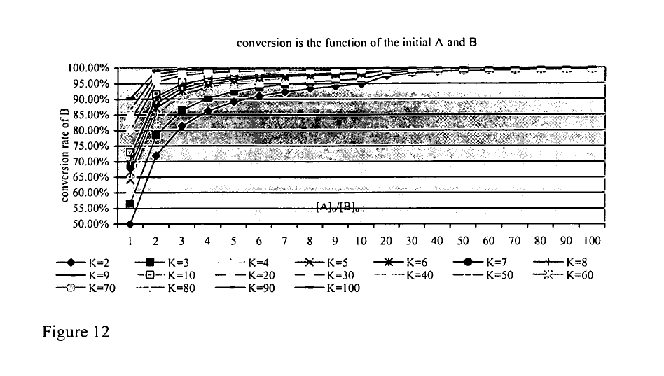

Defining the condition of [A]₀>>[B]₀: in chemical engineering, when the conversion rate is 99% or more, the conversion is said to be complete. Following this concept, for a reaction having K=2, it requires that [A]₀/[B]₀>=50 so that, [AB]_{Eq}/[B]₀>=99%. For a single probe-target pair, this seems satisfactory. Due to the wide span of the frequency of gene transcriptions in a cell (Greg Gibson and Spencer V. Muse<<A primer of genome science>>, pp 151-152), for a gene that has a thousand copies in the cell, 1% means 10 copies. It is still ten fold of a gene in which the transcription is at the frequency of one copy per cell. Potentially, this could interfere with the hybridization signals of genes with low frequency. Raising the ratio up to 1000/1, would further reduce cross hybridization, because the gene that has 1000 copies per cell, may have less than one copy left free in solution at equilibrium, which, theoretically can no longer interfere with the gene that has only one copy per cell. See FIG. 12.

5. Discussion

In the past years, the concept and the invention of the DNA microarray, together with the completion of the human genome project, has revolutionized our vision and the way doing biomedical or even the entire life science research. Use of the various existing microarray technologies has led to finding many meta genes. On the other hand, it has also been recognized that the technology is still considered to be in its infancy. The “noisiness” of microarray data has long been a topic in the field (Atul Butt, Nature Reviews Drug discovery vol. 1, Dec 2002; Erika Check, Nature 427, 91 (08 Jan. 2004)), and is almost always a problem in the use of microarrays. There are many causes of the “noise” in data in microarray experiments, such as biological variances, RNA sample preparation quality, defective array products, and the kinetics of fluorescence dyes activity, for example (Geoffrey J. McLachlan, Kini-Anh Do and Christophe Ambroise, <<Analyzing Microarray Gene Expression Data>>, 1.5.3, pp 18-19, 2004). However, these are common to any study involving these factors: just as in any scientific research, keeping experimental conditions consistent and maintaining standard procedures are always required, not just in DNA microarrays. These issues can be resolved by both standardization of experimental procedures and advances in the technology. Huge efforts have been invested to find clues to the mystery, and a solution (Zhijin Wu, Rafael A Irizarry. Nature Biotechnology 22, 656-658 (01 Jun. 2004); Li Zhang etc, Nature Biotechnology 22, 658 (2004); Ben Bolstad, “Probe-Level Analysis of Affymetrix GeneChip Microarray Data”, University of Minnesota, Minneapolis, Minn. Mar. 30, 2004 Minnesota Version. http://www.stat.berkeley.edu/users/bolstad; David B. Searls, Nature Reviews Drug Discovery 4, 45-58 (2005); J. Quackenbush. Science 302, 240 (07 Oct. 2003)). Many have postulated the standardization of microarray technology and the data (Lincoln Stein, Nature 417, 119-120 (09 May 2002); Nature 419, 323 (26 Sep. 2002); Joseph L. Hackett & Lawrence 3. Lesko, Nature Biotechnology 21, 742-743 (2003)). From the discussion herein, it is clearly demonstrated that the dream will not come true. Sharing of data and information among the research community can be realized only on the basis of gene expression level, but not signal intensity data. Designing DNA microarrays on the basis of chemical thermodynamics theoretically is one of the fundamental requirements, no matter what method is used to fabricate the array, or what type of array is used. The validation of the profiling result in “omics” scale has been called (Quackenbush. Nature Biotechnology 22, 613-614 (2004)). With the existing DNA microarray technology, it would be difficult, as the same data value can mean different gene expression levels, while the same gene expression level can produce different values of data. Although sometimes the results from RealTime PCR “confirm” those of microarrays, it is known that PCR amplifies the differences of any initial copy numbers, regardless of whether such are due to the gene expression level or to the sample concentration itself. There is no history of researchers “normnalizing” the amount of samples to add to a PCR reaction as the correct starting amount.

Obviously, standardization of experimental procedures and operations is important, and will improve DNA microarray data quality. Unfortunately, with the current DNA microarray technology these efforts are insufficient and have limited power to solve the existing problems.

As for data interpretation, the current DNA microarray technology and the data analysis systems can not resolve the problems discussed above due to the complicated relationship between the signal intensity and the abundance of each mRNA in the sample, which represents the true gene expression at transcription. To the extent that gene expression level is the object in a study, using the signal intensity as a direct measure of gene expression level will produce inaccurate or even incorrect information. Emerging technologies such as BioMEMS and other advances in nanotechnology may also support fabrication of smaller arrays with higher probe densities. The second generation of DNA microarrays is expected to be able to accomplish real gene expression profiling with higher efficiency and accuracy, and allow compiling of the obtained information into “omics” scale knowledge.

Part III: A Mathematical Model of Computation of the Absolute Gene Expression Level in the Microarray Data Analysis

This part of the invention involves a new microarray platform, and a data model for computational data analysis. In this model, a three dimensional data set is generated for each individual gene, which allows the computation of the absolute gene expression level (the mole/microgram RNA) of the gene. Such a model allows the building of a database for each individual sample, thus it is not always necessary to test samples and controls in parallel. The comparison analysis is conducted on real gene expression levels instead of on the experimental data. The comparison does not suffer from the variation of hybridization experiment conditions. Such a database allows the sharing of the gene expression information and will result in cost savings among the research community.

1. The principle

The DNA hybridization reaction, being a reversible chemical reaction, between a probe and the target can be expressed as 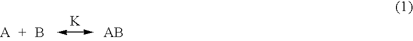
Here “A” represents the probe, “B” the target, “AB” the product, and “K” the chemical equilibrium constant.

After overnight hybridization reaction, the system reaches equilibrium. Then:
[AB]_{Eq}=K[A]_{Eq}[B]_{Eq} (2)
[AB]_{Eq}=K([A]₀−[AB]_{Eq})([B]₀−[AB]_{Eq}) (3)
If as a result of the microarray design the probe density is known, there are two variables left in equation (3): [B]₀ and K. [AB]_{Eq}, achieved by the signal intensity data, is available, but it needs to be converted into molar amount. In order to do so, an equivalent factor γ is also introduced:
Product in moles=[AB]_{Eq}*γ
γ represents one mole of [AB]_{Eq} equivalent to some certain units of signal intensity of the specific target sequence fragment that hybridizes to the probe. In total there are thus three variables. This means that to acquire the solutions for the three variables, at least three functions are required.

The experimental set up:

1. Develop a sample concentration series, such as:
[B]₀_{_{—}}₁:[B]₀_{_{—}}₂:[B]₀_{_{—}}₃=1:2:3 (or other ratios)
[B]₀_{_{—}}₁, [B]₀_{_{—}}₂ and [B]₀_{_{—}}₃ should be in proper range of sample concentration. Hybridize with the sample concentration of [B]₀_{_{—}}₁, [B]₀_{_{—}}₂ and [B]₀_{_{—}}₃ respectively at the same temperature, which insures that the chemical equilibrium constant K is equal for the three hybridizations. Then three data sets will be produced through the hybridization reactions: [AB]_{Eq}_{_{—}}₁, [AB]_{Eq}_{_{—}}₂ and [AB]_{Eq}_{_{—}}₃.
[AB]_{Eq}_{_{—}}₁,·γ=K([A]₀−[AB]_{Eq}_{_{—}}₁·γ)([B]₀_{_{—}}₁−[AB]_{Eq}_{_{—}}₁·γ) (4)
[AB]_{Eq}_{_{—}}₂,·γ=K([A]₀−[AB]_{Eq}_{_{—}}₂·γ)([B]₀_{_{—}}₂−[AB]_{Eq}_{_{—}}₂·γ) (5)
[AB]_{Eq}_{_{—}}₃,·γ=K([A]₀−[AB]_{Eq}_{_{—}}₃·γ)([B]₀_{_{—}}₃−[AB]_{Eq}_{_{—}}₃·γ) (6)
By combining equations (4), (5) and (6), the variables γ, K and [B]₀_{_{—}}₁ can be derived.
2. Three Dimensional Data Model

Gene expression is very complicated. Genome-wide gene expression profiling through DNA microarray hybridization is a highly complex system. As in Southern hybridization, cross hybridization always occurs. Since the fluorescence labeling is identical to all the target RNA/DNA molecules in the sample, it is difficult to differentiate whether the signal represents the specific target or was generated by cross hybridization. Hence when measuring a gene expression level with one probe, there is always the chance to obtain incorrect gene expression information. By making a concentration series as described above, a two dimensional data model can be built, which enables the computation of the absolute gene expression level. The two dimensional data model is in “one probe-one gene” platform of DNA microarray.

In this section, one more dimension is added to the data set model to enable verification of the computed gene expression level. In this data model, each gene is measured by multiple probes. In each single DNA microarray hybridization experiment, multiple data points are produced for each individual gene. Doing exactly the same computation for each point of data, a [B]₀ can be obtained. In an identical sample, the expression level of a gene is expected to be identical. The set of [B]₀ computed from the entire probe set (the probes that are designed to measure the same gene) supply the base to extract information establishing an accurate gene expression level.

Although identical results of [B]₀ are expected, due to the complexity of gene expression and the microarray hybridization reaction system, the results can be complicated. In reality, the following situations may happen: 1. the data is generated solely by hybridization with the specific target; 2. the data is generated mainly by the specific target with some interference; 3. serious cross hybridizations occurred, which account for a large fraction of the signal intensity; and 4. multiple cross hybridization reactions occur to the probe, while the hybridization to the specific target did not occur.

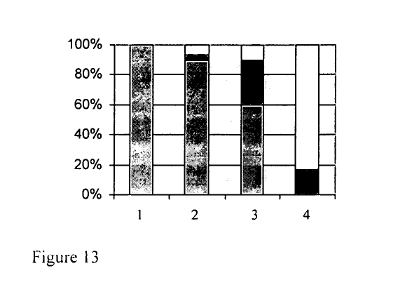

These situations are summarized in FIG. 13, in which situation 1 is ideal; the calculated [B]₀ is exactly the amount of the specific target; situation 2 is sort of noisy but still close; in situation 3, it is hard to deduce the amount of the specific target because the interference from cross hybridization is too great; while in the last situation the data is irrelevant to the specific target so that the result dos not reflect the level of [B]₀ at all.

In this model, one presumption is that the target is fragmented: there is no sharing of the target among probes—the length of each fragment allows hybridization to only one probe. Therefore the computed [B]₀ values are expected to be equal to each other. Although all the complexity exists, it is expected that the majority of the probes in a probe set have specificity for the specific target, i.e., their performance should be like 1 and 2 in FIG. 13.

For example, when 10 probes are used for a gene, there will be 10 values of [B]₀ being computed. If the majority of the 10 values, say more than 6 out of 10 are relatively centered around some certain value, we expect that the center value is probably close to the true expression level of the gene. The value distribution, the mean, median and standard deviation of the set of results, may supply hints for evaluation of the confidence on the gene expression level. The more centered the [B]₀ distribution is, the higher the probability that the median reflects the true [B]₀.

Combining section 1 and section 2, we can see that a three dimensional microarray data model of [AB]_{—}_{ⁱ}_{—}_{^{j}} for each gene is necessary to compute the absolute expression level of a gene. “_i” indicates the concentration series, “_j” the probe series.

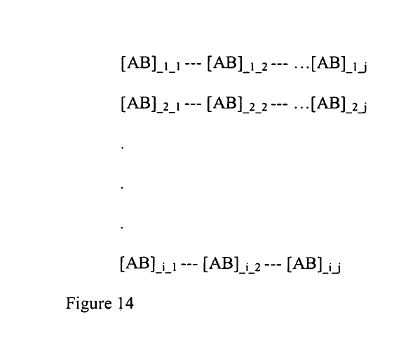

The data matrix in FIG. 14 illustrates the minimum requirement for the computation of the absolute expression level of one gene. The minimum “i” is 3, while there is no limit for “j”; 10 or more seems appropriate. The number of probes in a probe set should be large enough to allow an assessment of the probability that the computed [B]₀ is true or not, but it is not necessarily the case that more is better. Since each sample may contain thousands of genes in the hybridization reaction system, the chance of cross hybridization always exists. Too few probes may not result in confidence in the assessment.

3. How to Benefit from the Gene Expression Profiles

Gene expression profiling based on the data model in FIG. 10 outputs the absolute expression level of each individual gene. The computation of the gene expression level is based on the chemical equilibrium of each “probe-target” pair, and verified by using multiple “probe-target” pairs. Since the final result of the computation is the absolute gene expression level, the result can be stored in a database and is sharable unconditionally. For example, if a normal human lymphocyte sample is profiled and the result is stored in a database together with the raw data, when anyone else wants to do gene expression profiling of the same cell—human lymphocyte of a pathological condition or any other condition, he/she only needs to test the diseased sample, without a nornmal control sample. In the comparison of the two conditions, if a difference is found between the experimental sample and the control, there is the chance to trace back to the original raw data. Due to the chemical thermodynamics features of the reaction between each “probe-target”, the probe set data and the set of computed [B]₀ data display a certain pattern. If patterns of the entire probe set data and [B]₀ data display significant differences between the experimental sample and the control, this means that the probe set in the two arrays hybridized to different targets, thus there is serious interference from cross hybridization in one of the two samples. If only one or two probes' behavior are different, by removing the one or two probes, there is still the chance to verify the comparison results.

In the current common practice of DNA microarray experiments, usually there is only one array for one sample, and two can be used to compare two conditions of different samples. The three dimensional data model discussed above requires that a minimum of three arrays are used for each sample and on each array multiple probes are used to measure each individual gene, which seems on its face to be a more costly methodology. However, considering that the results of each data experiment can be stored in a database that can be shared, as a whole the research community will see lowered costs. In addition, the pattern of the computed [B]₀ set supplies information of interferences from cross hybridization. As the [B]₀ data accumulates, the gene expression information can be consolidated. This will help overcome the problem of inconsistency or “noisy” data in the current microarray technology, allowing the entire research community to combine their newly achieved knowledge from gene expression profiling studies and form new insights in molecular and cellular biology and life. As gene expression profiles as well as microarray data accumulate, the behavior of every probe set will be verified, and higher quality gene expression profiles can be achieved

What is claimed is:

1. An improved DNA microarray device comprising probes immobilized to the microarray surface, in which targets to be detected using the microarray are exposed to the probes to hybridize therewith, the improvement comprising providing on the microarray a quantity of a group of perfect match probes for a target of interest such that the molar ratio of each probe to the target fragment is at least about 20:1.

2. The improved DNA microarray device of claim 1 in which the ratio is at least about 50:1.

3. The improved DNA microarray device of claim 2 in which the ratio is at least about 1000:1.

4. The improved DNA microarray device of claim 1 in which the ratio is achieved at least in part by decreasing the target concentration in the sample being tested.

5. The improved DNA microarray device of claim 1 in which the microarray is incubated at a temperature of about a few degrees below the melting temperature of the hybridized probe-target pair.

6. The improved DNA microarray device of claim 1 further comprising using multiple probes to measure each single gene.

7. The improved DNA microarray device of claim 1 in which the at least about 20:1 ratio holds for every probe-target pair of interest.

8. The improved DNA microarray device of claim 1 in which the sample amount is sufficient such that the target gene with the lowest abundance produces a detectable signal after hybridization.

9. A method of determining the presence of a target sequence in a sample that is exposed to a microarray having perfect match probes coupled to its surface, comprising hybridizing under the same conditions a control sample and a test sample, with a series of perfect match probes available for the target sequence, and then comparing the measurements from both samples.

10. A method of determining the concentration of a target sequence in a sample that is exposed to a microarray having perfect match probes immobilized to its surface, comprising testing the sample and at least two different dilutions thereof under the same hybridization conditions, and comparing the data to determine the target concentration in the sample.

11. The method of claim 10 further comprising providing multiple probes for each target gene.

12. The method of claim 11 further comprising determining the target concentrations from each probe, and comparing the concentrations to determine a concentration value.

13. The method of claim 11 in which there are at least about ten probes for each target gene.

14. The method of claim 10 further comprising determining the identity of the target by coupling the concentration of the target with multiple physical chemical parameters of the hybridization reaction between each probe-target pair.

15. The method of claim 10 in which the ratio of sample dilutions is about 1:2:3.
